(19) United States
(12) Patent Application Publication
(10) Pub. No.: US 2008/0015577 A1
(43) Pub. Date: Jan. 17, 2008

(54) Spinal Correction Device

(76) Inventor: Alexander Loeb, New York, NY (US)

Correspondence Address: MORGAN & FINNEGAN, L.L.P., 3 WORLD FINANCIAL CENTER, NEW YORK, NY, 10281-2101, US

(21) Appl. No.: 11/456,830

(22) Filed: Jul. 11, 2006

(51) Int. Cl. A61F 2/30

(52) U.S. Cl. 606/61

(57) ABSTRACT

A spinal correction device for correcting scoliosis includes multiple implants that are each adapted to fit on an individual vertebra of the spine. Each implant includes a body and a protrusion extending from a face of the body. A hole in the body extends from a face of the body opposite to the face from which the protrusions extend. The protrusion extending from one of the implants interacts with the hole in an adjacent implant to permit relative longitudinal movement between the vertebrae that the two implants are respectively fixed to. The interaction of the protrusion and the hole, however, restricts relative horizontal movement between the two vertebrae.

### BACKGROUND

The present invention pertains to the field of devices for correcting scoliosis. Scoliosis is a condition in which the spine is laterally curved. In other words, a scoliotic spine displays curvature in a coronal plane, i.e., a plane that separates the body into forward and rear sections. The most common type of scoliosis is idiopathic scoliosis, which usually appears during puberty when the body is growing rapidly. The cause of idiopathic scoliosis is currently unknown.

FIG. 1 shows a normal spine 1 viewed from the back. The spine is made up of a series of bones known as vertebrae 2. The vertebrae can be divided into three separate regions: the cervical region 4, the thoracic region 5, and the lumbar region 6. There are seven cervical vertebrae that are known from top to bottom as C1-C7. Likewise, there are twelve thoracic vertebrae and five lumbar vertebrae that are known from top to bottom as T1-T12 and L1-L5, respectively. The lowermost lumbar vertebra L5 is connected to the sacrum 7. The opposite side of the sacrum from the side connected to the lowermost lumbar vertebra is connected to the tailbone 10. The sacrum sits in the pelvis, which is formed by upper 8 and lower 9 pelvic bones.

FIG. 2A shows a side view of three vertebrae 2 and FIG. 2B shows one of these vertebrae 2 as viewed from above. Intervertebral discs 11 are located between the vertebrae. Each vertebra has several large protrusions emanating from its backside. The large protrusion 12 that points straight back is known as the spinous process. Two transverse processes 120 on each vertebra extend away from the longitudinal line formed by the spinous processes on each vertebra. The transverse processes are connected to the spinous process by generally flat vertical surfaces known as laminae 121. Two pedicles 122 connect the transverse processes 120 to the vertebral body 123. The pedicles 122 and the laminae 121 constitute most of the vertebral arch that extends from the vertebral body and that forms a cavity 124 for the spinal cord (not shown) to pass through.

As shown in FIG. 1, a normal spine 1 is straight when viewed from the back. As shown in FIG. 3, however, a normal spine 1 is not straight when viewed from the side. A normal spine exhibits curvature in a sagittal plane, i.e., a plane that divides the body into left and right sections. The cervical 4 and lumbar 6 regions exhibit lordosis, i.e., they curve is such a manner that the concave side of the curve faces backwards. The thoracic region 5 exhibits kyphosis, i.e., it curves in such a manner that the concave part of the curve faces forward.

Unlike a normal spine, a scoliotic spine 100 is curved when viewed from the back as can be seen in FIG. 4. Additionally, the scoliotic spine is often twisted at the curve such that each vertebra in the curve is rotated with respect to its adjacent vertebrae.

Surgically-implanted devices for correcting scoliotic deformities are known. FIGS. 5A and SB show one such device, known as the Harrington system, as applied to a curve that affects a region spanned by thoracic vertebrae T4-T12 and lumbar vertebrae L1-L2. The Harrington system includes a distraction system and a compression system. The distraction system includes two metal hooks 13 and 14 that are respectively anchored to the vertebrae at the ends of the curve. As shown in FIG. 5A, hook 14 is anchored to vertebra L2 and hook 13 is anchored to vertebra T4. The hooks are connected to a rod 15 on the concave side of the curve. The upper end 16 of the rod 15 is notched to provide a ratcheted adjustment of the distance between the hooks 13 and 14. The compression system includes two hooks 17 and 18 and a rod 19 on the convex side of the curve. The hook 17 latches on to the top of a vertebra T6 that is part of the curve and the hook 18 latches on to the bottom of a vertebra T12 that is part of the curve. The rod 19 is threaded and the hooks 17 and 18 are connected to hex nuts 20a and 20b that are situated on the rod 19. The hooks 17 and 18 can be adjusted by the hex nuts 20a and 20b.

To correct a spinal curvature, a spreading instrument is used to adjust the distance between hooks 13 and 14, thereby stretching the curve on its concave side. At the same time, hooks 17 and 18 are adjusted by hex nuts 20a and 20b to compress the curve on its convex side. FIG. 5B shows a spine that has been straightened using Harrington instrumentation.

One major disadvantage of the Harrington system is that, because the distraction rod is essentially connected to only two vertebrae, the system will be completely inoperative if a failure occurs at one of the connection points. To overcome this problem, segmented devices that are connected to a number of the vertebrae that make up the curve have been developed. Some examples of segmented spinal instrumentation are Cotrel-Dubousset instrumentation, Zielke instrumentation and Texas Scottish Rite Hospital (TSRH) instrumentation. See generally An Atlas of Surgery of the Spine (Howard S. Rand & Lee H. Riley II eds., Lippincott-Raven 1998).

Another disadvantage of the Harrington and other known systems that employ rods is that the rods limit the ability of the vertebrae to move in the longitudinal direction. This limits the patent's ability to bend forward and also prevents normal growth of the patient's spine.

The Harrington system and most segmented systems for correcting scoliosis are used in conjunction with a bone graft to promote permanent fusion of the vertebrae that make up the scoliotic curve. The bone graft is made up of bone tissue that is removed from a donor site of the skeleton, such as the hip. The intervertebral discs between the vertebrae are removed and the bone tissue is inserted in their place. The bone graft inserted between the vertebrae will cause the vertebrae to fuse together after a period of time, usually six months or more. Spinal growth does not occur in a region where the vertebrae are fused together. Fusion is required because significant growth of the spine in the previously curved region while the Harrington system or a segmented system is in place reduces the system's effectiveness. The enlarged spine will regain some of its original scoliotic curve unless the Harrington or segmented system is repeatedly adjusted through surgical procedures. Therefore, fusion is required to stem the growth. Mobility is significantly restricted, however, when vertebrae of the spine are fused together. Mobility will be permanently restricted throughout the patient's lifetime, even though the tendency for the spine to regain a scoliotic curve significantly decreases with adulthood. Additionally, spinal fusion that occurs during or before puberty may reduce overall height or cause the body trunk to be disproportionately sized relative to the rest of the body.

A few spinal correction devices that do not promote fusion are known. One such device is disclosed in U.S. Pat. No. 5,951,555 to Rehak et al. (“Rehak”). The device disclosed in Rehak includes segments that are fixed to successive vertebrae. See Rehak FIG. 1. Each segment includes a spring that fits within a bearing case in an adjacent segment. See Rehak col. 3,1. 64-col. 4,1. 6, col. 6,1. 30-55. The springs are fixed to one segment at an angle of 45° to the vertical vertebrae axis and received in bearing cases on adjacent segments also at an angle of 45° to the longitudinal vertebrae axis. See Rehak col. 3,1. 52-55, col. 8,1. 37-40. Because the springs and bearing cases are set at an angle of 45° to the vertical vertebrae axis in the device disclosed in Rehak, the springs and corresponding bearing cases do not allow for longitudinal motion between adjacent segments while restricting horizontal motion. Moreover, the cyclical loading on the springs that would result from typical movements of the spine can result in the failure of one or more of the springs due to fatigue. The device disclosed in U.S. Pat. No. 5,672,175 to Martin et al. (“Martin”) also has a complicated construction that relies on the elasticity of a number of springs to allow mobility and growth of the spine. As such, the device disclosed in Martin has the same defects as the device disclosed in Rehak.

The plate and rod system disclosed in Ye et al., “A New Spinal Instrumentation Without Fusion for the Treatment of Progressive Idiopathic Scoliosis in Growing Children,” Journal of Musculoskeletal Research No. 3 and 4 (2003) 201-09 relies on a sliding connection between the two rods of the system and the two hooks at the lower end of the scoliotic curve to allow mobility and growth of the spine. This plate and rod system, however, suffers from a significant defect of the Harrington system: because it is only connected to the spine at a minimal number of points, i.e., because it is not segmented, failure of the system at one of the connection points will completely prevent the system from performing its function.

What is needed in the art is a spinal correction device for correcting a scoliotic condition that is only temporarily affixed to the spine, that does not promote permanent fusion of the spinal vertebrae that make up the curve, that allows for longitudinal movement between adjacent vertebrae to facilitate back-bending and spinal growth, and that has a simple yet sturdy construction that is not subject to failure.

### SUMMARY

An object of the invention is to provide a spinal correction device that corrects a scoliotic condition without promoting permanent fusion of the vertebrae that make up the curve.

A further object of the invention to provide a spinal correction device that is designed to be removed after spinal growth has stopped.

A still further object of the invention is to provide a spinal correction device that is simple in construction but that is capable of restricting relative horizontal movement between the vertebrae that make up the curve while allowing relative longitudinal movement between these vertebrae.

A still further object of the invention is to provide a spinal correction device that straightens a scoliotic curve but retains the natural kyphosis and lordosis of the spine.

These and other objects of the invention are accomplished by a spinal correction device comprising a plurality of implants that are each adapted to fit on an individual vertebra of the spine. Each implant includes a body and a protrusion extending from a face of the body. A hole in the body extends from an opposite face of the body. The protrusion extending from one of the implants interacts with the hole in an adjacent implant to permit relative longitudinal movement between the vertebrae that the two implants are respectively fixed to. The interaction of the protrusion and the hole, however, restricts relative horizontal movement.

In a further aspect of the invention, the protrusion on one of the implants may be curved to help the spine maintain its natural kyphosis and lordosis of the spine.

The spinal correction device is designed for temporary application. It can be removed after spinal growth has stopped and it does not promote permanent fusion of the vertebrae that make up the curve.

### BRIEF DESCRIPTION OF THE DRAWINGS

Additional aspects, features, and advantages of the invention, both as to its structure and operation, will be understood and will become more readily apparent when the invention is considered in light of the following description made in conjunction with the accompanying drawings, wherein:

FIG. 1 shows a normal spine as viewed from the back.

FIG. 2A shows three vertebrae of a spine as viewed from the side.

FIG. 2B shows a typical thoracic vertebra as viewed from above.

FIG. 3 shows a normal spine as viewed from the side.

FIG. 4 shows a spine exhibiting scoliosis as viewed from the back.

FIGS. 5A and 5B show a known device for correcting a scoliosis.

FIGS. 6A and 6B are rear and side views of a spinal correction device embodying the invention attached to a patient's spine.

FIG. 6C is a view of the section at cutting plane 6C-6C in FIG. 6A.

FIG. 7 is an elevated view of the spinal correction device embodying the invention shown in FIG. 6.

FIG. 8 is an exploded view of the spinal correction device embodying the invention shown in FIGS. 6 and 7.

FIG. 9 is a side view of one implant of the spinal correction device in FIGS. 6-8 showing one possible configuration of a protrusion-receiving internal hole.

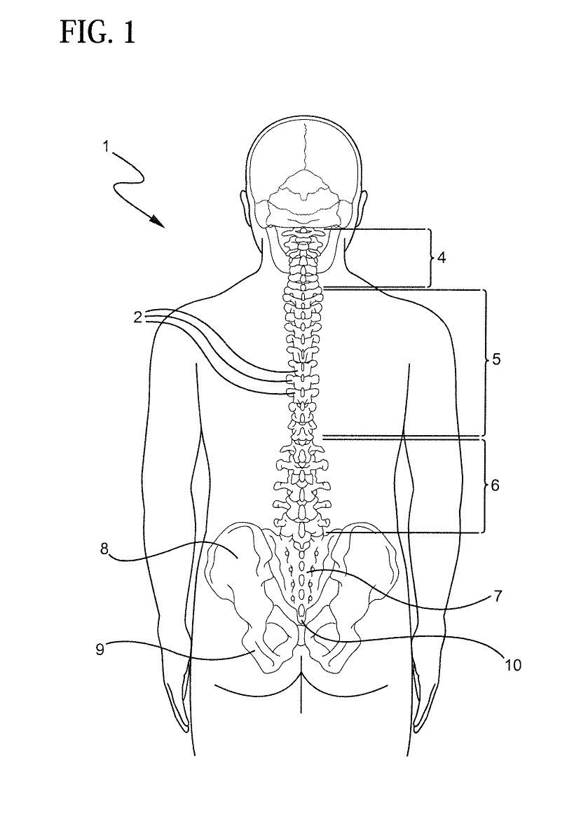

### DETAILED DESCRIPTION

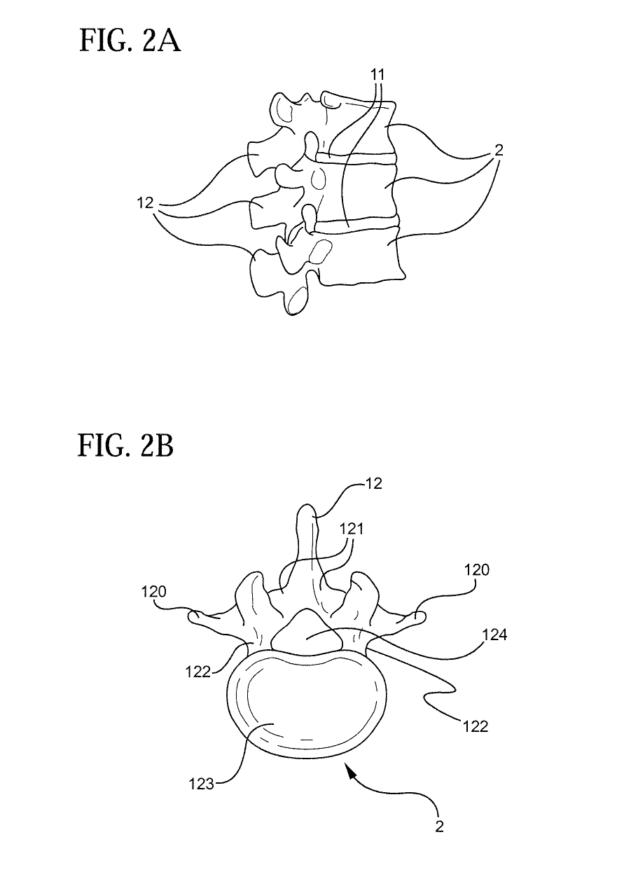

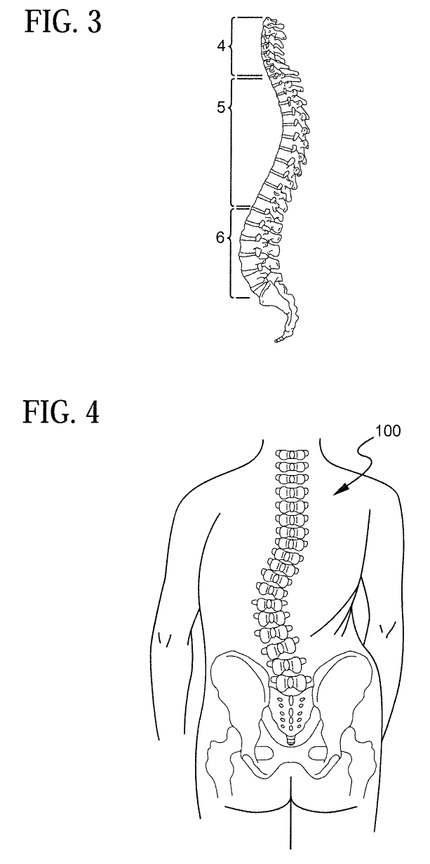

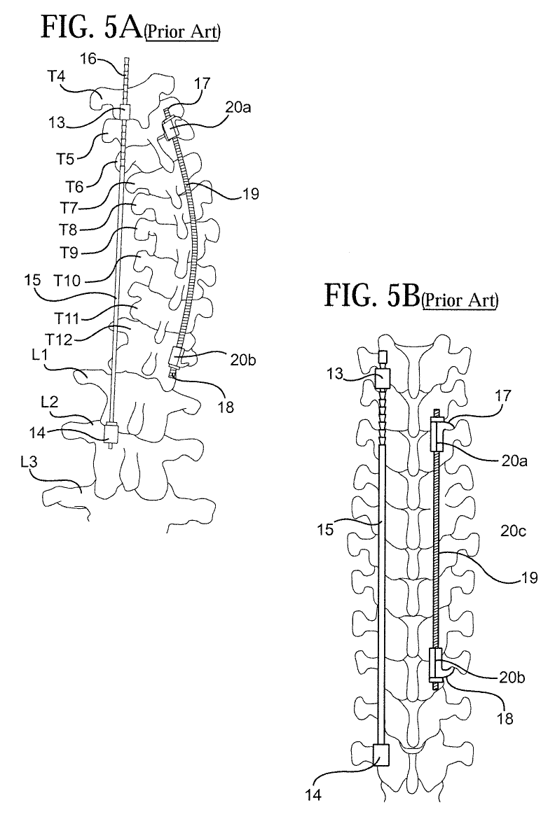

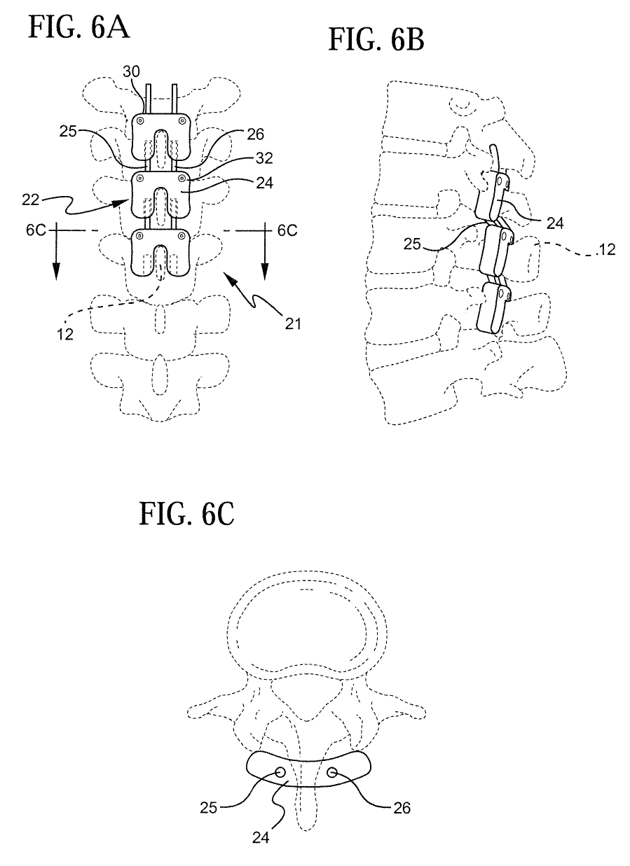

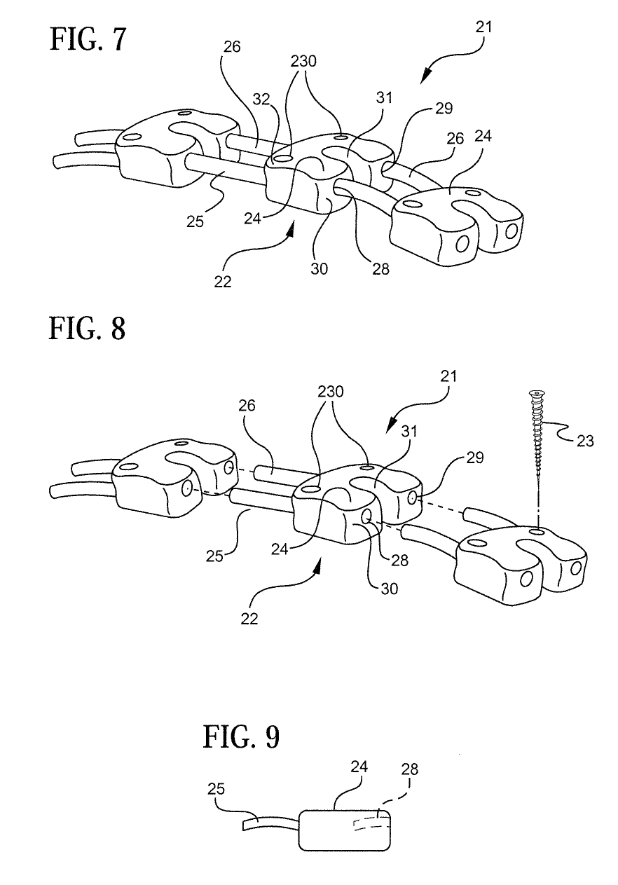

FIGS. 6-8 show a spinal correction device 21 embodying the invention that is comprised of a plurality of implants 22. Each implant is adapted to fit onto and be connected to an individual vertebra that is part of a scoliotic curve of a spine. As shown in FIGS. 6-8, each implant includes a body 24. In the embodiment shown in the figures, two protrusions 25 and 26 extend from one face 27 of the body. The protrusions preferably have circular cross-sections. The cross-section of the protrusion may alternatively be tubular. The protrusions for a particular implant may be curved or straight. FIGS. 7 and 8 show both curved and straight protrusions. The body 24 contains two holes 28 and 29. The holes 28 and 29 begin on a second face 30 of the body that is opposite to the first face 27 of the body. The holes 28 and 29 extend substantially in the same direction as the protrusions 25 and 26 and terminate in the interior portion of the body. As shown in FIGS. 6A-6C, the body 24 of each implant contains a central opening 31 that allows the implant to be mounted around the spinous process 12 of the vertebra to which it is attached. The central opening 31 penetrates the second face 30 of the body so that the body is essentially “horseshoe shaped.” The implant is connected to a vertebra using screws 23, which are received in through-holes 230 in screw-receiving sections 32. In the embodiment depicted in FIGS. 6-8, the screw-receiving sections 32 are a portion of the body 24. The screws contact the vertebra at the laminae 121 (FIG. 2B).

The implants are preferably attached to the vertebrae so that the protrusions are on the top side of their respective implants, i.e., the side of the implants that faces the patient's head when the implants are affixed to the spine, and the holes are on the bottom side of the respective implants. When the implants are attached to the spinal vertebrae, the two protrusions 25 and 26 on one of the implants are received in and interact with the two holes 28 and 29 on the implant above it to form two sliding hinges, i.e., the protrusions slide within the holes. The holes 28 and 29 have a shape that generally conforms to the shape of the protrusions they receive, i.e., if the protrusions are curved, the holes are also curved. FIG. 9, for example, shows a hole 28 in the body 24 that receives a curved protrusion. The interaction of the protrusions and holes allow for longitudinal movement of the vertebrae relative to each other while resisting the horizontal forces acting on the vertebrae that formerly formed the scoliotic curve, thereby restricting relative horizontal movement between these vertebrae. The protrusions have a strength sufficient to resist horizontal forces on these vertebrae without bending significantly.

Although three implants are shown in the FIGS. 6-8, the actual number of implants will depend on the number of vertebrae that are a part of a particular patient's scoliotic curve. Each of the implants has essentially the same features. The two implants at the ends of the scoliotic curve, however, may have slightly different features, because each of these two implants only interacts with one other implant instead of two. For example, an implant at the end of the scoliotic curve may have protrusions but no holes or holes but no protrusions.

Additionally, although the implants have essentially the same components, each implant or one or more of its features may be shaped and/or sized according to the particular vertebra to which the implant is to be attached and the location of that vertebra in the scoliotic curve. For example, while the two protrusions 25 and 26 extending from any one implant will have the same shape, the protrusions on different implants may have different configurations. As mentioned, the protrusions 25 and 26 can be curved or straight. Curved protrusions can have a selected radius of curvature. The curved protrusions shown in FIGS. 7-9 have their concave sides facing down as viewed in these figures, i.e., the concave sides would face the vertebrae when the implants are affixed to them. Alternatively, the curved protrusions can have concave sides that face up as viewed in FIGS. 7-9. Straight protrusions can be orthogonal to the first face 27, or they can be inclined, i.e., non-orthogonal, with respect to the first face 27. If inclined, the protrusions extend from the body 24 at a selected angle of inclination. The shapes and orientations of the protrusions of the implants are chosen such that the spine retains its natural kyphosis and lordosis when the spinal correction device is installed. This assures the spine's sturdiness, shapeliness and uninhibited cushioning function in the future. As already mentioned, the holes receiving the protrusions, which are in a different implant, will have a shape that generally conforms with the shape of the protrusions. Therefore, in general, the design of any one implant in the device will influence or be influenced by the design of an implant adjacent to the one implant when the device is affixed to the spine. The size of each implant will also be determined in part by the overall size of the spine to which the implants are attached, i.e., whether the spine is that of a small child or a teenager.

Although each implant in the embodiment of the invention shown in FIGS. 6-8 is connected to its respective vertebra with two screws 32, it may alternatively be affixed to the vertebra by any suitable fastening or adhering means, for example, a single screw or an adhesive such as bone cement. Additionally, if a screw is used to affix the implant, the screw can penetrate the vertebra at one of the pedicles instead of one of the laminae. Multiple screws can be used to attach the implant to the vertebra at, e.g., both pedicles, or to one or more of the laminae in addition to one or more of the pedicles. The location, shape, and size of the screw-receiving section 32 can be modified depending on what part of the vertebra the implant is to attached to, e.g., a lamina or a pedicle. While the screw-receiving sections 32 in the embodiment shown in FIGS. 6-8 are portions of the body 24, they may alternatively be separate components, e.g., flanges that extend from the body.

It is contemplated that a series of standard implants can be available to the surgeon. In other words, the surgeon can choose from a series of standard implant models. Each model would have a particular size and would have protrusions that have a particular radius of curvature, if curved, or a particular angle of inclination, if straight. For any one implant size, there would be multiple models, each model having a different radius of curvature or angle of inclination for the protrusions. The surgeon would choose an implant model for each vertebra in the curve. For example, for a vertebra in a teenage spine at the apex of the thoracic kyphosis, the surgeon can select an implant model that is large in size and has protrusions with a large radius of curvature. While the implant model the surgeon chooses for any one vertebra may not be as optimally sized and configured as a custom-made implant, it would suitably perform the required function at a fraction of the cost of a custom-made implant.

Each implant is composed of a strong metallic alloy, for example, a titanium alloy or stainless steel. The protrusions and the holes are covered in a material that functions as an artificial cartilage so as to lubricate the back and forth motion of the implants with each other.

The implants are affixed to the vertebrae that make up the curve through a surgical procedure. In addition to installing the implants to the spine, the surgeon will initially straighten the spine during this procedure. A variety of methods can be used to straighten the spine and install the implants. One method is to use one of the known spinal instrumentations to straighten the spine and then to affix the implants. For example, Harrington rods can be used to straighten the spine prior to the affixation of the implants. The implants can be properly affixed to the vertebrae even when the Harrington rods are present because the rods are positioned farther away from the central spinous processes than the lateral ends of the implants. Once the implants are affixed to the vertebrae, the Harrington rods can be removed.

Another method is to move each vertebra in the curve individually to the proper position and then to affix an implant to it. Facetectomies are normally required prior to the performance of this method to allow the vertebrae to be individually manipulated. The steps of manipulating a vertebra and then affixing an implant to it are repeated until an implant is affixed to every vertebra that was a part of the curve. Alternatively, for certain curves, the entire curve can be straightened at once by manipulating the rib cage. The implants can then be affixed to the vertebrae forming the curve while the rib cage is held in the position that straightens the curve.

The spinal correction device can, for example, be affixed to the spine during a surgical procedure during the teenage or pre-teenage years of a patient with scoliosis. While the device is affixed to the spine it allows sufficient mobility of the spine. The patient can bend his or her back forward and backward without substantial restriction. Additionally, unlike spinal fusion, the spinal correction device does not inhibit growth of the spine. The protrusions 25 and 26 have a length that ensures that they continue to interact with the holes 28 and 29 when the longitudinal distance between the vertebrae increases due to forward bending or spinal growth. The device can be adjusted through a subsequent surgery if growth of the spine requires one or more implants to be repositioned. Ordinarily no more than one such adjustment surgery should be required. Additionally, the spinal correction device is designed to be removed once spinal growth has stopped, when there is little risk that the deformation will return. Because the device does not promote fusion, once the device is removed, full mobility of the spine is restored and there are little to no risks concerning sports or osteoporosis. Therefore, unlike spinal fusion, the device does not permanently restrict the mobility of the scoliosis patient.

Although the present invention has been described in considerable detail with reference to certain preferred embodiments thereof, other embodiments are possible. For example, although each implant in the described embodiment of the invention includes two protrusions and two holes, each implant can, instead, include one protrusion and one hole. Alternatively, each implant can include more than two protrusions and more than two holes. Additionally, although the protrusions extend from the top side of each implant in the described embodiment, the implants can alternatively be attached to the vertebrae so that the protrusions are on the bottom side of their respective implants. Therefore, the spirit and scope of the appended claims should not be limited to the description of the preferred versions contained herein.

What is claimed is:

1. A spinal correction device comprising:
at least two implants, each implant being adapted to fit onto a spinal vertebra, each implant comprising:
a body;
a protrusion extending from a first face of the body;
a hole in the body, the hole extending from a second face of the body, the second face of the body being opposite to the first face of the body;
wherein the protrusion of one of the at least two implants interacts with the hole in a second implant of the at least two implants when the spinal correction device is fixed to a spine to permit relative longitudinal movement between the one vertebra and the adjacent vertebra but restrict horizontal movement between the one vertebra and the adjacent vertebra.

2. The spinal correction device according to claim 1, wherein the protrusion of at least one implant of the at least two implants is curved.

3. The spinal correction device according to claim 1, wherein each protrusion is curved and each protrusion has a radius of curvature that leaves the spine with its natural kyphosis and lordosis.

4. The spinal correction device according to claim 1, wherein the protrusion of at least one implant of the at least two implants is straight.

5. The spinal correction device according to claim 1, wherein the protrusion of at least one implant of the at least two implants is not orthogonal with the first face.

6. The spinal correction device according to claim 1, wherein the protrusion of at least one implant of the at least two implants has a circular cross-section.

7. The spinal correction device according to claim 1, wherein the protrusion of at least one implant of the at least two implants has a tubular cross-section.

8. The spinal correction device according to claim 1, wherein at least one implant of the at least two implant comprises two protrusions and at least one implant of the at least two implants comprises two holes.

9. The spinal correction device according to claim 1, wherein each implant of the at least two implants is made of a strong metallic alloy.

10. The spinal correction device according to claim 1, wherein the protrusion and the hole of each implant of the at least two implants are covered in a material that functions as an artificial cartilage.

11. A spinal correction device according to claim 1, further comprising:
a screw for each implant of the at least two implants, the screw adapted to connect the implant to a spinal vertebra.

12. A method for installing a spinal correction device comprising at least two implants to a spine exhibiting scoliosis, the spine exhibiting scoliosis comprising multiple vertebrae and having a region along its length that is curved when viewed from the backside of the spine, the method comprising:
connecting at least one rod to the spine;
adjusting the spine using the at least one rod so that it appears straight when viewed from the backside of the spine;
affixing the at least two implants to adjacent vertebrae that were located in the curved region of the spine;
disconnecting the at least one rod from the spine.
